(19) United States
(12) Patent Application Publication
(10) Pub. No.: US 2023/0355466 A1
(43) Pub. Date: Nov. 9, 2023

(54) MASSAGER

(71) Applicant: Shuting Cheng, Heyuan City (CN)

(72) Inventor: Shuting Cheng, Heyuan City (CN)

(21) Appl. No.: 18/355,780

(22) Filed: Jul. 20, 2023

(30) Foreign Application Priority Data
Jun. 6, 2023 (CN) 2023214337246

(51) Int. Cl. A61H 23/02; A61F 7/00

(52) U.S. Cl. CPC A61H 23/0254; A61F 7/007; A61H 2201/0192; A61H 2201/14; A61H 2201/0207; A61H 2201/0221; A61F 2007/0078

(57) ABSTRACT

The disclosure provides a massager. The massager includes a telescopic rotary driver, where a movable member, a first gear, a second gear and a driving mechanism are arranged inside a fixed cavity of the telescopic rotary driver, a matching structure of the telescopic rotary driver is connected to a connecting structure, the connecting structure is connected to an elastic massage sleeve, thereby reducing the volume of the telescopic rotary driver. An electric heating element or a vibrator is arranged on the elastic massage sleeve, thereby realizing effect of vibration or heating during massage while completing telescopic motion and rotation.

### TECHNICAL FIELD

The disclosure pertains to the technical field of massage devices, and specifically pertains to a massager.

### BACKGROUND

At present, massagers in the prior art are generally telescopic massage devices, which generally drive massage inner bladders to perform reciprocating motion through motors and crank connecting rod mechanisms, but this type of massagers has general massage effect. Therefore, a telescopic rotary massager capable of synchronously driving its rotation in the process of driving an inner bladder thereof to make reciprocating translational motion has been developed, such as a telescopic rotary massager with a patent announcement number of CN204050242U. However, when being driven for telescopic rotary, this type of products shall be provided with two motors distributed front and rear, such structure makes the volume of a mechanism that drives an inner bladder of the massage to make telescopic rotary motion larger, resulting in an overall low space utilization of the massager. At the same time, when an electric heating sheet or a vibrating motor are mounted on the inner bladder of the massager, a battery and a controller need to be further configured, which makes the massager more complex and the manufacture more difficult.

### SUMMARY

In order to overcome the above defects in the prior art, the disclosure provides a massager.

The massager designed by the disclosure includes a telescopic rotary driver and a matching structure. The telescopic rotary driver includes a fixed cavity, a massage member is mounted on the mounting part of the matching structure, a movable member, a first gear, a second gear and a driving mechanism are arranged inside the fixed cavity, the driving mechanism is mounted on the movable member, the first gear and the second gear mesh with each other and are respectively mounted on the movable member in a rotational manner through a rotating shaft, and the driving part of the driving mechanism is in transmission connection with the first gear; an elongated groove is formed on the cavity wall of the fixed cavity in a width direction of the fixed cavity, a limiting member is arranged at the eccentric position of the first gear, and the limiting member is placed inside the elongated groove; the movable member is connected to the cavity wall of the fixed cavity through a guide structure arranged in a length direction of the fixed cavity, so that the movable member can be reciprocated in the length direction of the fixed cavity; the second gear includes a gear part and a connecting structure formed at the center of the gear part, the gear part meshes with the first gear for transmission, and a conductive member is arranged on the connecting structure; a circuit board is arranged on the matching structure, a central conductive layer and a conductive ring layer located inside the central conductive layer are formed on the surface of the circuit board, the central conductive layer and the conductive ring layer are spaced apart from each other and arranged concentrically, an electronically controlled massage auxiliary member with vibration and heating functions is arranged on the massage member, and the central conductive layer and the conductive ring layer are electrically connected to the electronically controlled massage auxiliary member, respectively; the connecting portion and the connecting structure of the matching structure are connected to each other, and the two separate conductive probes of the conductive member are butted against and electrically connected to the central conductive layer and the conductive ring layer, respectively.

According to the massager described above, the connecting structure is a connecting sleeve, the connecting portion of the matching structure is a matching sleeve, the matching sleeve is sleeved on the connecting sleeve, and the matching structure is connected to the connecting structure through a fastening structure; the circuit board is arranged inside the matching sleeve, and the conductive member is arranged at the port of the connecting sleeve or inside the connective sleeve.

According to the massager described above, the movable member includes a horizontal outer wall surface and a vertical end surface, the first gear is mounted on the horizontal outer wall surface in a rotational manner through a rotating shaft, and the second gear is mounted on the vertical end surface in a rotational manner through a rotating shaft; the connecting sleeve is sleeved on a rotating shaft on the vertical end surface used for mounting the second gear in a rotational manner, a rotating shaft on the vertical end surface for mounting the second gear in a rotational manner is hollow to form a transitional channel, and a conductive member is arranged inside the transitional channel.

According to the massager described above, the matching sleeve is formed by surrounding a plurality of elastic pieces arranged in an annular array, the fastening structure includes snap fasteners connected on the outer wall of the connecting sleeve and snap slots arranged on the inner wall of at least one elastic piece and corresponding to positions of the snap fasteners, and the snap fasteners are snap-fitted on the snap grooves.

According to the massager described above, the fastening structure includes positioning grooves connected on the outer wall of the connecting sleeve and positioning grooves arranged on the inner wall of at least one elastic piece and corresponding to positions of the positioning protrusions, and the snap fasteners are embedded in positioning grooves.

According to the massager described above, a mounting part of matching structure is a cup body, the massage member is an elastic massage sleeve, the elastic massage sleeve is positioned inside the cup body, an electric heating sheet is arranged on the outer wall or peripheral side portion of the elastic massage sleeve, and positive and negative electrode conducting wires of the electric heating sheet are electrically connected to the central conductive layer and the conductive ring layer, respectively.

According to the massager described above, the massager further includes a supporting housing, the elastic massage sleeve is arranged inside the supporting housing, an elastic massage ring is arranged at the port of the supporting housing, the elastic massage ring and the elastic massage sleeve are coaxially arranged, a vibrator is implanted inside the peripheral side portion of the elastic massage ring, a conductive connecting member is fixed onto the inner wall of the supporting housing, the battery inside the telescopic rotary driver is electrically connected to the conductive connecting member through the controller, and the conductive connecting member is electrically connected to the vibrator through an electric connecting wire.

According to the massager described above, the driving mechanism includes a first motor, and a rotating shaft of the first motor is in transmission connection with the first gear.

According to the massager described above, the driving mechanism further includes a worm gear, a first worm and a third gear, the third gear and the first gear mesh with each other, the first worm is mounted on a rotating shaft of the motor, the third gear and the worm gear are distributed up and down and mounted on the movable member in a rotational manner through a rotating shaft, the worm gear and the first worm mesh with each other, and the first worm is coaxially arranged on the rotating shaft of the first motor.

According to the massager described above, the driving mechanism further includes a second motor and a second worm, the first worm and the second worm are arranged parallel to each other, the second worm meshes with the worm gear for transmission, the second worm is mounted on a rotating shaft of the second motor, the second worm is coaxially arranged on the rotating shaft of the second motor.

According to the massager described above, a limiting member is mounted on the vertical end surface, a gear part is limited between the limiting ring body and the vertical end surface, and the connecting structure passes through the inner hole of the limiting ring body.

According to the massager described above, the massager further includes a shell body, on which a fixed cavity and an opening communicating with the fixed cavity are formed, the connecting sleeve protrudes from the opening of the shell body, a battery and a controller are arranged inside the shell body, the electric connecting wire of the conductive member is connected to the controller after passing through the transitional channel, the movable member and a through hole in the housing, and the battery is connected to the controller.

The massager designed in the disclosure has the following beneficial effects:
1. The first gear is mounted on the movable member in a rotational manner to drive the rotation of the first gear, so that the limiting member arranged in the eccentric position of the first gear moves in the elongated groove, and the movable member is driven to reciprocate in an axial direction under the guidance of the guide structure, at the same time, the first gear meshes with the second gear to drive the second gear to rotate, and an elastic massage sleeve can be mounted on the second gear to drive the elastic massage sleeve to rotate, so that only one set of the driving mechanism for driving the rotation of the first gear can realize a purpose that the elastic massage sleeve performs rotary motion while making telescopic motion, so as to reduce the volume of the mechanism that drives the elastic massage sleeve of the massager for making telescopic rotary motion, and improve the overall space utilization of the massager.
2. The central conductive layer and the conductive ring layer are separately butted against the conductive probes of the conductive member, so that the electric heating sheet can drive the rotation of the massage member while generating heating, and the electric connecting wire is prevent from being entangled and other phenomena. The only one set of the battery and the controller can simultaneously control telescopic rotary motion of the massage member and heating of the electric heating sheet.
3. The electric heating sheet is arranged on the outer wall or peripheral side portion of the elastic massage sleeve, which promotes blood circulation better during massage and achieves a better effect of massage.
4. The elastic massage ring is arranged at the port of the supporting housing, which increases the length of the elastic massage sleeve.
5. The matching sleeve is snap-fitted on the connecting sleeve through the snap fasteners, which facilitates the disassembly of a massage group composed of the supporting housing, the elastic massage sleeve and an elastic massage ring from the telescopic rotary driver, so that the massager is convenient for carrying.

### BRIEF DESCRIPTION OF THE DRAWINGS

FIG. 1 is an overall structural schematic diagram (1) of a massager according to the disclosure;

FIG. 2 is an exploded view (1) of a massager according to the disclosure;

FIG. 3 is a structural schematic diagram (1) of a telescopic rotary driver according to the disclosure;

FIG. 4 is a structural schematic diagram (2) of a telescopic rotary driver according to the disclosure;

FIG. 5 is an exploded view (2) of a massager according to the disclosure;

FIG. 6 is an exploded view (3) of a massager according to the disclosure;

FIG. 7 is a structural schematic diagram of an elongated groove according to the disclosure;

FIG. 8 is a structural schematic diagram of a connecting structure according to the disclosure;

FIG. 9 is a structural schematic diagram of an elastic massage ring according to the disclosure;

FIG. 10 is an exploded view of a supporting housing according to the disclosure;

FIG. 11 is an overall structural schematic diagram (2) of a massager according to the disclosure;

FIG. 12 is an exploded view of a telescopic rotary driver according to the disclosure;

FIG. 13 is a full sectional view (1) of the massager according to the disclosure; and

FIG. 14 is a full sectional view (2) of the massager according to the disclosure.

In the accompanying drawings: supporting housing 1, half part shell 10, lower half part 11, upper half part 12, end cover 13, conductive connecting member 14, telescopic rotary driver 2, movable member 20, horizontal outer wall surface 201, vertical end face 202, rotating shaft 203, transitional channel 204, first gear 21, second gear 22, connecting structure 220, gear 30 part 221, connecting sleeve 222, snap fastener 223, positioning groove 224, limiting member 23, limiting ring body 24, guide structure 25, elongated guide groove 251, elongated guide rail 252, first motor 26, second motor 27, first worm 28, second worm 29, elastic massage sleeve 3, worm gear 30, elastic massage protrusion 31, third gear 32, matching structure 4, cup body 41, matching sleeve 42, elastic piece 421, snap slot 422, positioning protrusion 423, elastic massage ring 5, cover plate 6, conductive member 7, charging interface 8, circuit board 9, central conductive layer 91, conductive ring layer 92, controller 101, battery 102, controller button 103, and elongated groove 104.

### DESCRIPTION OF THE EMBODIMENTS

### Embodiment

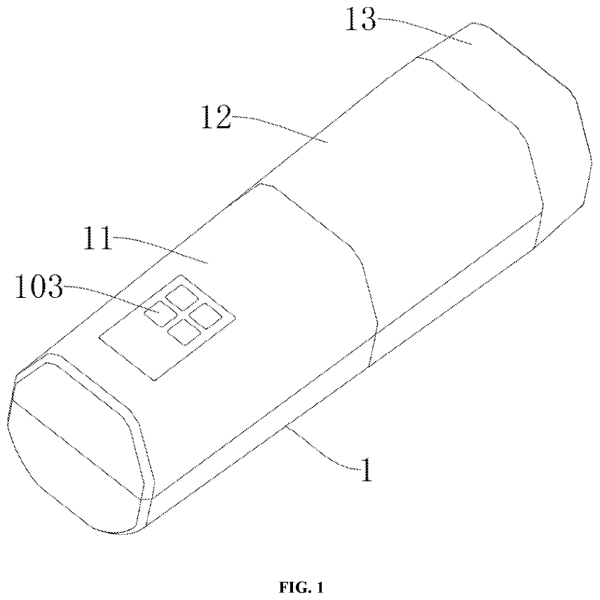

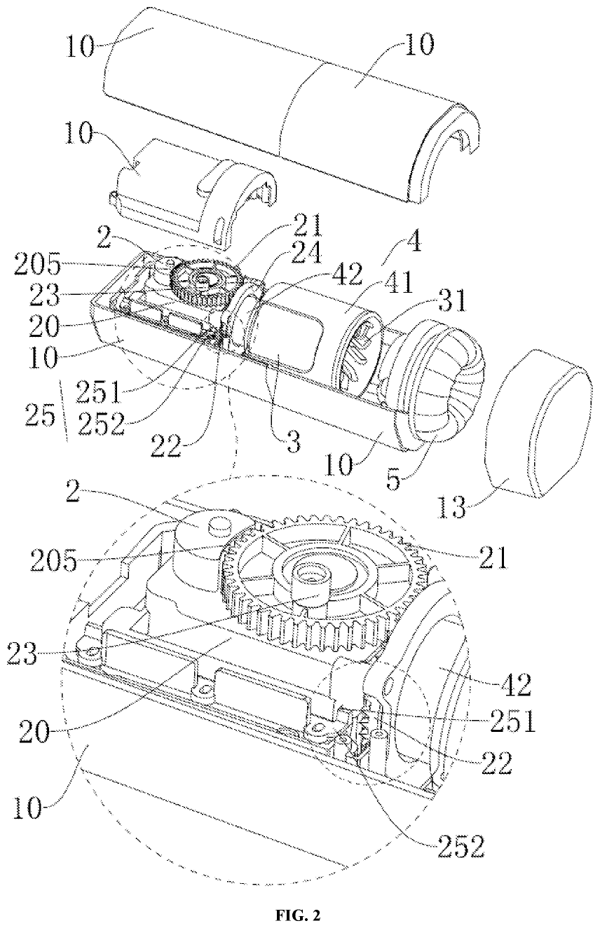

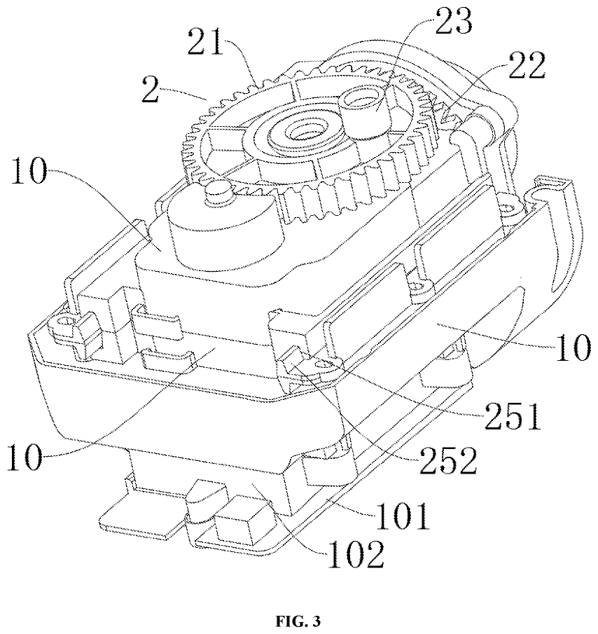

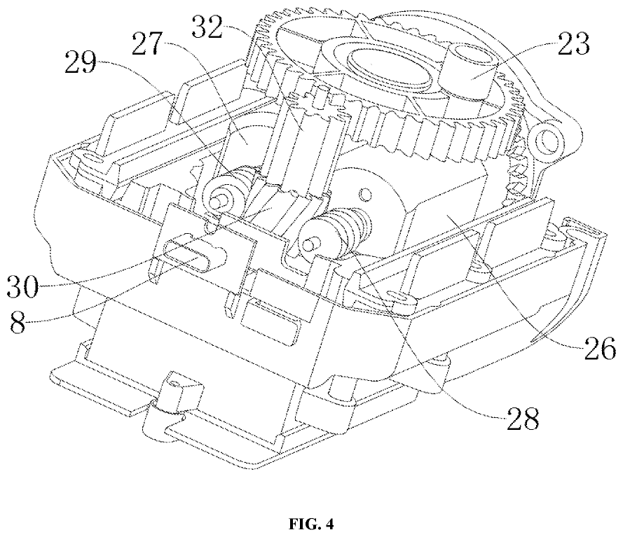

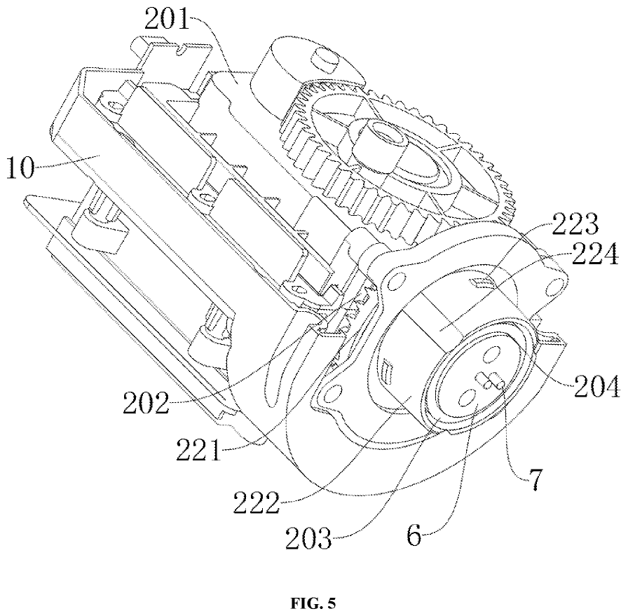

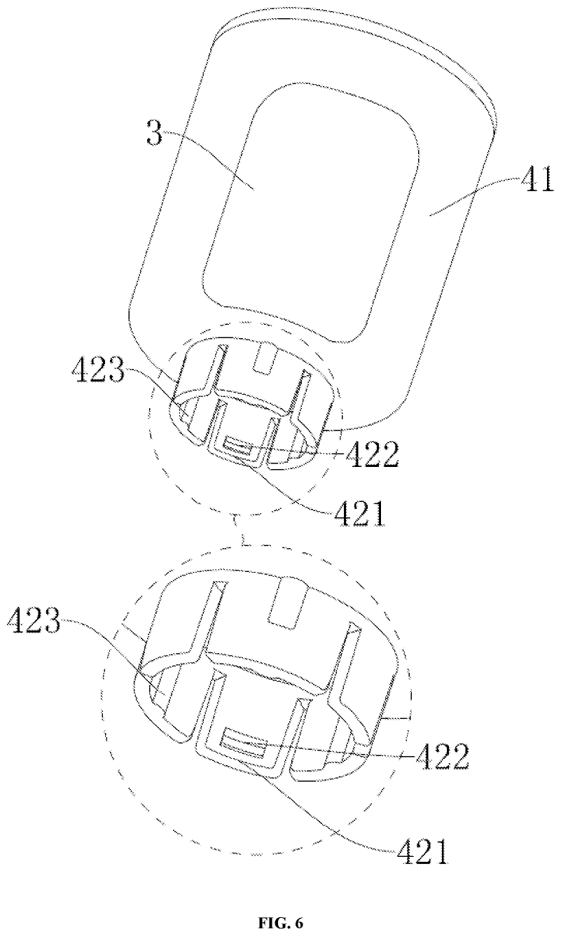

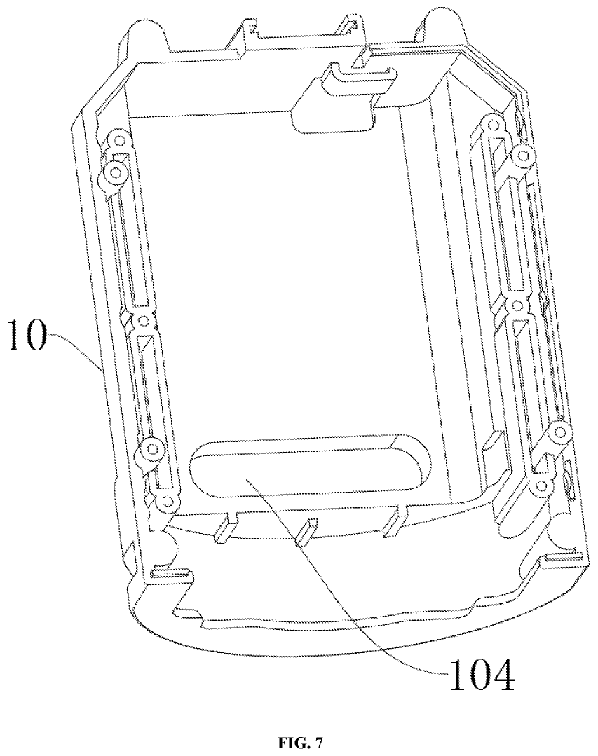

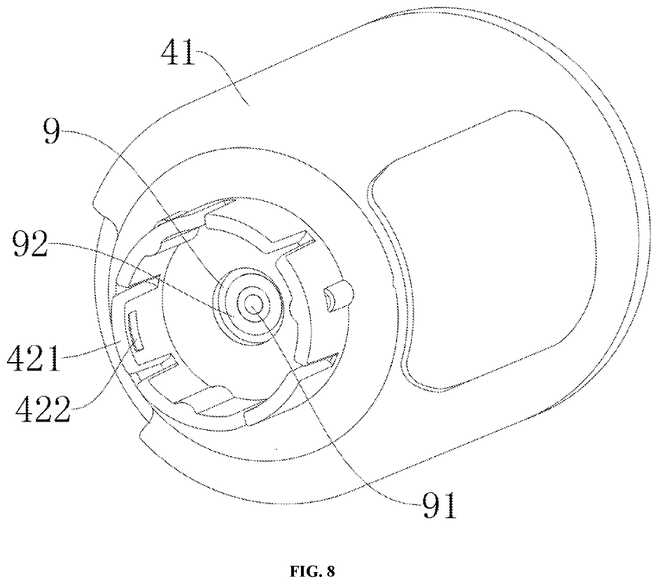

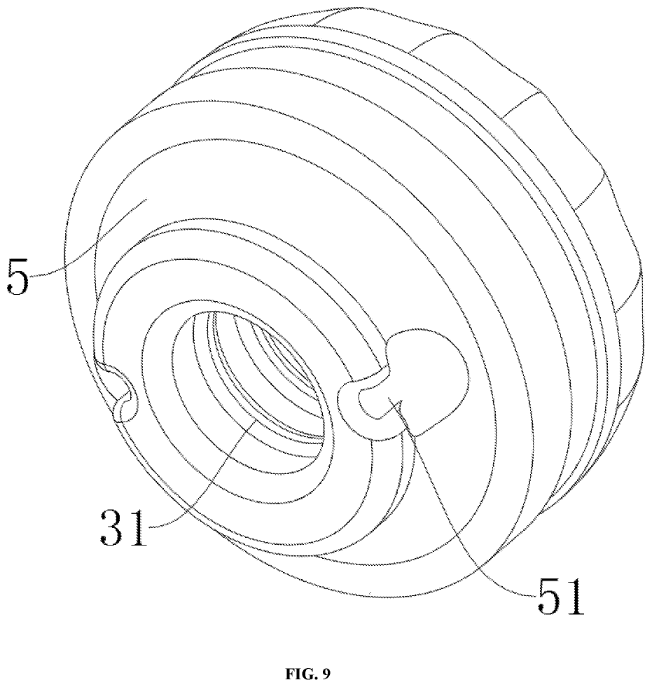

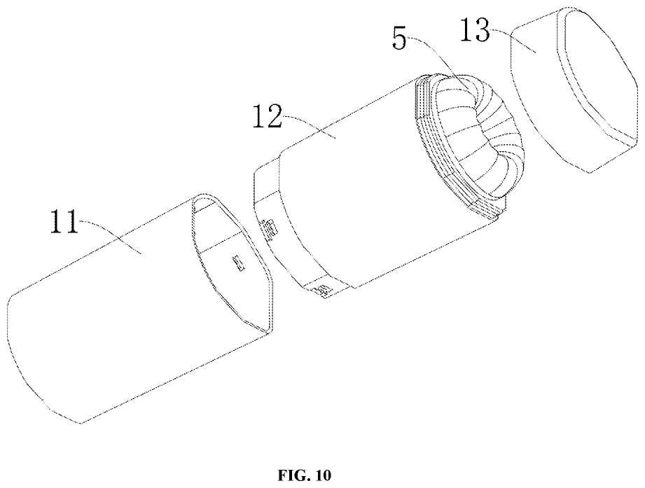

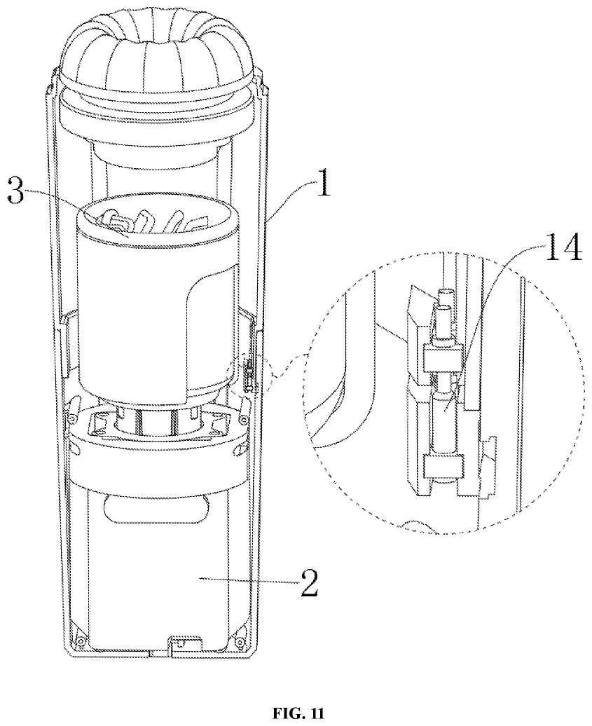

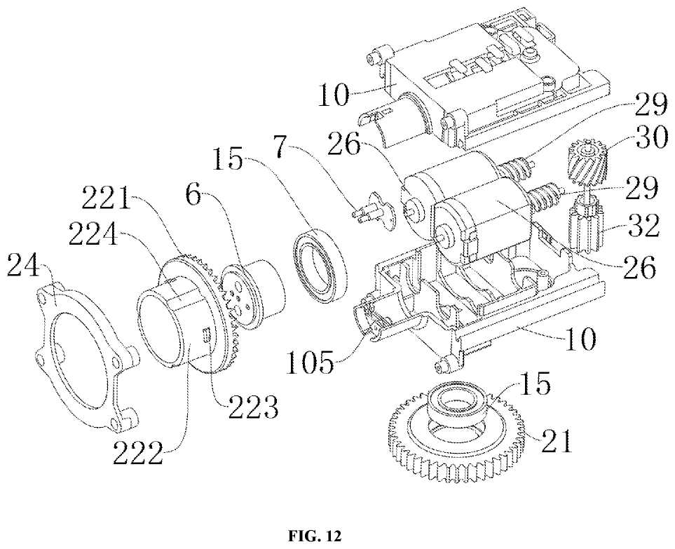

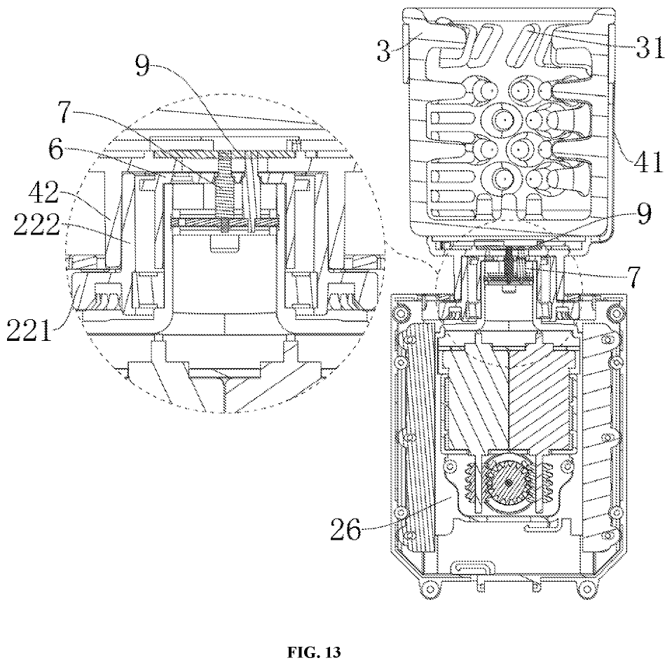

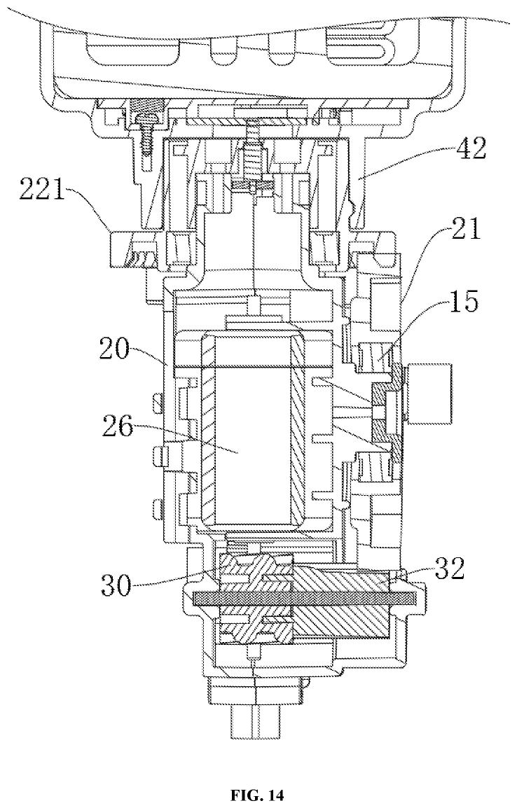

As shown in FIGS. 1-14, a massager described in this embodiment is configured to massage the user's body, especially sensitive parts of the body, or may also be used to massage fingers.

The massager includes a supporting housing 1 and a telescopic rotary driver 2. The telescopic rotary driver 2 includes a shell body, on which a fixed cavity and an opening communicating with the fixed cavity are formed. A movable member 20, a first gear 21, a second gear 22, a driving mechanism, a battery 102 and a controller 101 are arranged inside the fixed cavity, the first gear 21 and the second gear 22 mesh with each other and are respectively mounted on the movable member 20 in a rotational manner through a rotating shaft, that is, a bearing 15 is sleeved at a position of a horizontal outer wall surface 201 of the movable member 20 through a rotating shaft, the first gear 21 is sleeved on an outer ring of the bearing 15, the first gear and the bearing are closely matched with each other, and the second gear 22 is mounted on a vertical end face 202 of the movable member 20 in a rotational manner through a rotating shaft to realize perpendicular of the first gear 21 and the second gear 22; an elongated groove 104 is formed on the cavity wall of the fixed cavity in a width direction of the fixed cavity, a limiting member 23 is arranged at the eccentric position of the first gear 21, the limiting member 23 is placed inside the elongated groove 104, and the limiting member 23 is a roller mounted in the eccentric position of the first gear 21 in a rotational manner through a rotating shaft, so that the limiting member 23 moves more smoothly in the elongated groove; the movable member 20 is connected to the cavity wall of the fixed cavity through a guide structure 25 arranged in a length direction of the fixed cavity, so that the movable member 20 can be reciprocated in the length direction of the fixed cavity. The second gear 22 includes a gear part 221 and a connecting structure 220 formed at the center of the gear part 221, and the gear part 221 meshes with the first gear 21 for transmission.

The driving mechanism includes a first motor 26, a worm gear 30, a first worm 28 and a third gear 32, the third gear 32 and the first gear 21 mesh with each other, the first worm 28 is mounted on a rotating shaft of the motor, the third gear 32 and the worm gear 30 are distributed up and down and mounted on the movable member 20 in a rotational manner through a rotating shaft, the worm gear 30 and the first worm 28 mesh with each other, and the first worm 28 is coaxially arranged on the rotating shaft of the first motor 26; the first worm 28 is driven by energizing the first motor 26 to be meshed with the worm gear 30 for transmission, at the same time, the third gear 32 is driven to rotate and then the first gear 21 is driven to rotate, so that the limiting member 23 arranged in the eccentric position of the first gear 21 moves in the elongated groove, and the movable member 20 is driven to reciprocate in an axial direction under the guidance of the guide structure 25, at the same time, the first gear 21 meshes with the second gear 22 to drive the second gear 22 to rotate, and an elastic massage sleeve 3 can be mounted on the second gear 22 to drive the elastic massage sleeve 3 to rotate, so that the elastic massage sleeve 3 can realize a purpose of rotary motion while making telescopic motion.

The driving mechanism further includes a second motor 27 and a second worm 29, the first worm 28 and the second worm 29 are arranged parallel to each other, the second worm 29 meshes with the worm gear 30 for transmission, the second worm 29 is mounted on a rotating shaft of the second motor 27, the second worm 29 is coaxially arranged on the rotating shaft of the second motor 27, the first motor 26 and the second motor 27 are arranged parallel to each other and side by side, and the worm gear 30 is driven to rotate through rotation in the same direction of the first worm 28 and the second worm 29, so that the rotary speed and stability of rotation of the worm gear 30 are improved, and the battery 102 inside the telescopic 20 rotary driver 2 is electrically connected to the first motor 26 and the second motor 27 respectively through the controller 101.

The guide structure 25 includes two elongated guide rails 252 and two elongated guide grooves 251 arranged in the length direction of the fixed cavity, the elongated guide rails 252 are arranged inside the elongated guide grooves 251 in a sliding manner, the elongated guide rails 252 can be fixed onto the elongated side inner wall of the fixed cavity, the elongated guide grooves 251 can be formed on the elongated side outer wall of the movable member 20, or the elongated guide rails 252 can be fixed onto the elongated side outer wall of the movable member 20, and the elongated guide grooves 251 can be formed on the elongated side outer wall of the fixed cavity. The guide structure 25 can also adopt a structure in which a linear guide rail cooperates with a slider, or a structure in which a guide rod cooperates with a slider.

The elastic massage sleeve 3 is arranged inside the supporting housing 1, a matching structure 4 is mounted on the end portion of the elastic massage sleeve 3 facing the telescopic rotary driver 2, the matching structure 4 is fixedly connected to the connecting structure 220 through a fastening structure, a plurality of elastic massage protrusions 31 are formed on the inner wall of the elastic massage sleeve 3, and the elastic massage sleeve 3 is made of silicone material, so it has elasticity. When a body part to be massaged is placed into the elastic massage sleeve 3, the massage protrusions are in close contact with the external wall of the body part to be massaged under the action of elasticity, and the elastic massage sleeve 3 performs the telescopic rotary motions, so that friction massage is performed between the body part to be massaged and the massage protrusions.

The connecting structure 220 is a connecting sleeve 222, the bearing 15 is sleeved on a rotating shaft on a vertical end surface 202 used for mounting the second gear 22 in a rotational manner, the connecting sleeve 222 is sleeved on the outer ring of the bearing 15, the connecting sleeve and the bearing are closely matched with each other, a rotating shaft 203 on the vertical end surface 202 for mounting the second gear 22 in a rotational manner is hollow to form a transitional channel 204, a conductive member 7 is arranged inside the transitional channel 204, a cover plate 6 is fixed at the port of the transitional channel 204, and two conductive probes of the conductive member 7 separately protrude through the cover plate 6; and a circuit board at the rear end of the conductive member is positioned in a fixing groove 105 of the transitional channel 204.

The matching structure 4 includes a cup body 41 and a matching sleeve 42 formed at the end portion of the cup body 41, and the elastic massage sleeve 3 is positioned in the cup body 41 to position and mount the elastic massage sleeve 3, so that the stability of the elastic massage sleeve 3 in the supporting housing 1 can be improved. The connecting sleeve 222 protrudes from the opening of the shell body, the matching sleeve 42 is sleeved on the connection sleeve 222, a circuit board 9 is arranged inside the matching sleeve 42, a central conductive layer 91, and a conductive ring layer 92 located inside the central conductive layer 91, are formed on the surface of the circuit board 9, the central conductive layer 91 and the conductive ring layer 92 are spaced apart from each other and arranged concentrically, and the two separate conductive probes of the conductive member 7 are butted against and electrically connected to the central conductive layer 91 and the conductive ring layer 92, respectively; an electric heating sheet is arranged on the outer wall or peripheral side portion of the elastic massage sleeve 3, positive and negative electrode conducting wires of the electric heating sheet are electrically connected to the central conductive layer 91 and the conductive ring layer 92, respectively, the battery 102 inside the telescopic rotary driver 2 is electrically connected to the conductive member 7 through the controller 101, where the electric connecting wire of the conductive member 7 is connected to the controller 101 after passing through the transitional channel 204, the movable member 20 and a through hole in the housing, the battery 102 is connected to the controller 101, and the electric heating sheet can also be replaced by a vibrating motors , so as to realize heat or vibration during massage.

Snap fasteners 223 and positioning grooves 224 are formed on the outer wall of the connecting sleeve 222, the matching sleeve 42 is formed by surrounding a plurality of elastic pieces 421 arranged in an annular array, snap slots 422 corresponding to positions of the snaps 223, and positioning protrusions 423 corresponding to the positioning grooves 224, are formed on inner walls of the elastic pieces 421, the snap fasteners 223 are snap-fitted on the snap slots 422, and the positioning protrusions 423 are embedded in positioning grooves 224; the structural setting thereof facilitates the disassembly of a massage group composed of the supporting housing 1, the elastic massage sleeve and an elastic massage ring 5 from the telescopic rotary driver 2, so that the massager is convenient for carrying, and the plurality of elastic pieces 421 are combined with the cup body 41 to form an integrated plastic structure.

The peripheral side portion of the elastic massage sleeve 3 has a sheet cavity, the electric heating sheet is placed inside the sheet cavity, the sheet cavity can be two and arranged opposite one another, and the electric heating sheet can be a graphene electric heating sheet, or a flexible circuit board heating sheet composed of an electric heating wire.

The elastic massage ring 5 is arranged at the port of the supporting housing 1. The elastic massage ring 5 is made of silica gel material and has certain elasticity, and the plurality of elastic massage protrusions 31 are formed on the inner wall of the elastic massage ring, a body part to be massaged can be inserted into the elastic massage sleeve 3 after passing through an inner hole of the elastic massage ring 5, the elastic massage ring 5 and the elastic massage sleeve 3 are coaxially arranged, a vibrator is implanted inside the peripheral side portion of the elastic massage ring 5, a conductive connecting member 14 is fixed onto the inner wall of the supporting housing 1, the battery 102 inside the telescopic rotary driver 2 is electrically connected to the conductive connecting member 14 through the controller 101, the conductive connecting member 14 is electrically connected to the vibrator through an electric connecting wire, and the vibrator can perform vibration massage on the body part outside the opening of the elastic massage sleeve 3, so that the massage area increases. The vibrator is a vibration motor with eccentric rotation, and annular protrusions on the inner wall of the port of the supporting housing 1 are correspondingly embedded in annular grooves of the outer wall of the elastic massage ring 5, so as to mount the elastic massage ring 5 in a fixed manner, the port of the supporting housing 1 is covered with an end cover 13 to cover the elastic massage ring 5, and the conductive connecting member 14 is fixed in the fixing groove on the inner wall of the supporting housing 1.

A limiting ring body 24 is fixed through bolts on the vertical end surface 202, a gear part 221 is limited between the limiting ring body and the vertical end surface 202, and the connecting structure 220 passes through the inner hole of the limiting ring body 24, so as to position the gear part 221.

The movable member 20 further includes a mounting cavity and a meshing port 205 communicating with the mounting cavity, the first motor 26, the second motor 27, the worm gear 30, the first worm 28, the second worm 29 and the third gear 32 are all arranged inside the mounting cavity, and the meshing portion of the first gear 21 meshes with the third gear 32 through the meshing port. The controller 101 is composed of a main control module and control buttons 103. The main control module can be a CPU chip or an MCU chip, and a program for controlling the first motor 26, the second motor 27, the electric heating sheet and the vibrator is built-in; the supporting housing 1 is formed by an upper half part 12 and a lower half part 11 in an insertion manner, the shell of the telescopic rotary driver 2 is fixed inside the inner cavity of the lower half part 11, the matching structure 4 and the elastic massage sleeve 3 are located inside the inner cavity of the upper half part 12, the elastic massage ring 5 is mounted on the port of the upper half part 12, and the upper half part 12 and the lower half part 11 of the supporting housing 1, the movable member 20 and the shell thereof each is composed of two symmetrically arranged half part shells 10.

The charging interface 8 is connected to the battery to charge the battery, and the aperture of the elastic massage sleeve and the aperture of the elastic massage ring are matched with the body part to be massaged, so that the inner wall of the elastic massage sleeve and the elastic massage ring is in close contact with the body part to be massaged.

What is claimed is:

1. A massager, comprising a telescopic rotary driver and a matching structure, wherein the telescopic rotary driver comprises a fixed cavity, a massage member is mounted on the mounting part of the matching structure, a movable member, a first gear, a second gear and a driving mechanism are arranged inside the fixed cavity, the driving mechanism is mounted on the movable member, the first gear and the second gear mesh with each other and are respectively mounted on the movable member in a rotational manner through a rotating shaft, and the driving part of the driving mechanism is in transmission connection with the first gear;
an elongated groove is formed on the cavity wall of the fixed cavity in a width direction of the fixed cavity, a limiting member is arranged at the eccentric position of the first gear, and the limiting member is placed inside the elongated groove;
the movable member is connected to the cavity wall of the fixed cavity through a guide structure arranged in a length direction of the fixed cavity, so that the movable member can be reciprocated in the length direction of the fixed cavity;
the second gear comprises a gear part and a connecting structure formed at the center of the gear part, the gear part meshes with the first gear for transmission, and a conductive member is arranged on the connecting structure;
a circuit board is arranged on the matching structure, a central conductive layer and a conductive ring layer located inside the central conductive layer are formed on the surface of the circuit board, the central conductive layer and the conductive ring layer are spaced apart from each other and arranged concentrically, an electronically controlled massage auxiliary member with vibration and heating functions is arranged on the massage member, and the central conductive layer and the conductive ring layer are electrically connected to the electronically controlled massage auxiliary member, respectively; and
the connecting portion and the connecting structure of the matching structure are connected to each other, and the two separate conductive probes of the conductive member are butted against and electrically connected to the central conductive layer and the conductive ring layer, respectively.

2. The massager according to claim 1, wherein the connecting structure is a connecting sleeve, the connecting portion of the matching structure is a matching sleeve, the matching sleeve is sleeved on the connecting sleeve, and the matching structure is connected to the connecting structure through a fastening structure; the circuit board is arranged inside the matching sleeve, and the conductive member is arranged at the port of the connecting sleeve or inside the connective sleeve.

3. The massager according to claim 2, wherein the movable member comprises a horizontal outer wall surface and a vertical end surface, the first gear is mounted on the horizontal outer wall surface in a rotational manner through a rotating shaft, and the second gear is mounted on the vertical end surface in a rotational manner through a rotating shaft; the connecting sleeve is sleeved on a rotating shaft on the vertical end surface used for mounting the second gear in a rotational manner, a rotating shaft on the vertical end surface for mounting the second gear in a rotational manner is hollow to form a transitional channel, and a conductive member is arranged inside the transitional channel.

4. The massager according to claim 2, wherein the matching sleeve is formed by surrounding a plurality of elastic pieces arranged in an annular array, the fastening structure comprises snap fasteners connected on the outer wall of the connecting sleeve and snap slots arranged on the inner wall of at least one elastic piece and corresponding to positions of the snap fasteners, and the snap fasteners are snap-fitted on the snap grooves.

5. The massager according to claim 4, wherein the fastening structure comprises positioning grooves connected on the outer wall of the connecting sleeve and positioning grooves arranged on the inner wall of at least one elastic piece and corresponding to positions of the positioning protrusions, and the snap fasteners are embedded in positioning grooves.

6. The massager according to claim 4, wherein a mounting part of matching structure is a cup body, the massage member is an elastic massage sleeve, the elastic massage sleeve is positioned inside the cup body, an electric heating sheet is arranged on the outer wall or peripheral side portion of the elastic massage sleeve, and positive and negative electrode conducting wires of the electric heating sheet are electrically connected to the central conductive layer and the conductive ring layer, respectively.

7. The massager according to claim 6, wherein the massager further comprises a supporting housing, the elastic massage sleeve is arranged inside the supporting housing, an elastic massage ring is arranged at the port of the supporting housing, the elastic massage ring and the elastic massage sleeve are coaxially arranged, a vibrator is implanted inside the peripheral side portion of the elastic massage ring, a conductive connecting member is fixed onto the inner wall of the supporting housing, the battery inside the telescopic rotary driver is electrically connected to the conductive connecting member through the controller, and the conductive connecting member is electrically connected to the vibrator through an electric connecting wire.

8. The massager according to claim 1, wherein the driving mechanism comprises a first motor, and a rotating shaft of the first motor is in transmission connection with the first gear.

9. The massager according to claim 8, wherein the driving mechanism further comprises a worm gear, a first worm and a third gear, the third gear and the first gear mesh with each other, the first worm is mounted on a rotating shaft of the motor, the third gear and the worm gear are distributed up and down and mounted on the movable member in a rotational manner through a rotating shaft, the worm gear and the first worm mesh with each other, and the first worm is coaxially arranged on the rotating shaft of the first motor.

10. The massager according to claim 9, wherein the driving mechanism further comprises a second motor and a second worm, the first worm and the second worm are arranged parallel to each other, the second worm meshes with the worm gear for transmission, the second worm is mounted on a rotating shaft of the second motor, the second worm is coaxially arranged on the rotating shaft of the second motor.

11. The massager according to claim 3, wherein a limiting member is mounted on the vertical end surface, a gear part is limited between the limiting ring body and the vertical end surface, and the connecting structure passes through the inner hole of the limiting ring body.

12. The massager according to claim 2, wherein the massager further comprises a shell body, on which a fixed cavity and an opening communicating with the fixed cavity are formed, the connecting sleeve protrudes from the opening of the shell body, a battery and a controller are arranged inside the shell body, the electric connecting wire of the conductive member is connected to the controller after passing through the transitional channel, the movable member and a through hole in the housing, and the battery is connected to the controller.
